(19) United States
(12) Patent Application Publication
(10) Pub. No.: US 2018/0221332 A1
(43) Pub. Date: Aug. 9, 2018

(54) Flash-Melt Cannabinoid Formulations

(71) Applicant: CannTab Therapeutics Limited, Toronto (CA)

(72) Inventors: Jeff Renwick, Thornhill (CA); Joshi Laxminaraya, Ajax (CA)

(21) Appl. No.: 15/888,407

(22) Filed: Feb. 5, 2018

Related U.S. Application Data
(60) Provisional application No. 62/454,830, filed on Feb. 5, 2017.

(51) Int. Cl. A61K 31/352; A61K 9/20; A61K 47/10; A61K 47/69; A61K 47/26

(52) U.S. Cl. CPC A61K 31/352; A61K 9/2081; A61K 47/26; A61K 47/6923; A61K 47/10

(57) ABSTRACT

The present invention relates to dosage forms for the production of flash-melt cannabis resin and extract oral dosage forms

### FIELD OF THE INVENTION

The present invention relates to dosage forms for the production of flash-melt cannabis resin and extract oral dosage forms. In addition to one or more cannabinoids, the dosage forms include a drug-ion exchange resin complex comprising a core composed of a cannabinoid complexed with a pharmaceutically acceptable ion-exchange resin. Methods of making and products containing this complex are described. Dosage forms may also include other conventional ingredients such as sweetening and flavoring agents. The subject formulations are advantageous in that they are good tasting, stable dosage forms, and the tablets prepared therefrom on conventional equipment disintegrate in the mouth in quickly.

### BACKGROUND OF THE INVENTION

Cannabinoids are a class of diverse chemical compounds that act on cannabinoid receptors on cells that repress neurotransmitter release in the brain. The most notable cannabinoid is the phytocannabinoid tetrahydrocannabinol (THC), the primary psychoactive compound of cannabis. Cannabidiol (CBD) is another major constituent of the plant. There are at least 85 different cannabinoids isolated from cannabis, exhibiting varied effects. From Wikipedia http://en.wikipedia.org/wiki/Tetrahydrocannabinol accessed May 25, 2015. All or any of these cannabinoids can be used in the present invention.

Synthetic cannabinoids encompass a variety of distinct chemical classes: the cannabinoids structurally related to THC, the cannabinoids not related to THC, such as (cannabimimetics) including the aminoalkylindoles, 1,5-diarylpyrazoles, quinolines, and arylsulfonamides, and eicosanoids related to the endocannabinoids. All or any of these cannabinoids can be used in the present invention.

Delta-9-Tetrahydrocannabinol (dronabinol) is a naturally occurring compound and is the primary active ingredient in marijuana. Marijuana is dried hemp plant Cannabis Sativa. The leaves and stems of the plant contain cannabinoid compounds (including dronabinol). Dronabinol has been approved by the Food and Drug Administration for the control of nausea and vomiting associated with chemotherapy and for appetite stimulation of patients suffering from wasting syndrome. Synthetic dronabinol is a recognized pharmaceutically active ingredient, but natural botanical sources of cannabis rather than synthetic THC are also known in the art. All or any of these cannabinoids can be used in the present invention.

Dronabinol is a light yellow resinous oil that is sticky at room temperature and hardens upon refrigeration. Dronabinol is insoluble in water and is formulated in sesame oil. It has a pKa of 10.6 and an octanol-water partition coefficient: 6,000:1 at pH 7. After oral administration, dronabinol has an onset of action of approximately 0.5 to 1 hours and peak effect at 2 to 4 hours. Duration of action for psychoactive effects is 4 to 6 hours, but the appetite stimulant effect of dronabinol may continue for 24 hours or longer after administration.

Dronabinol is the international nonproprietary name for a pure isomer of THC, (−)-trans-Δ⁹-tetrahydrocannabinol, which is the main isomer, and the principal psychoactive constituent, found in cannabis. Synthesized dronabinol is marketed as Marinol (a registered trademark of Solvay Pharmaceuticals).

Marinol is manufactured as a gelatin capsule containing synthetic delta-9-tetrahydrocannabinol (THC) in sesame oil. It is taken orally and is available in 2.5 mg, 5 mg and/or 10 mg dosages. Marinol is prescribed for the treatment of cachexia in patients with AIDS and for the treatment of nausea and vomiting associated with cancer chemotherapy in patients who have failed to respond adequately to conventional antiemetic treatments. Like other oils provided in gelatin dosage forms, there is an urgent need for solid (powder and tablet) dosage forms of this drug as provided in the instant invention.

Despite FDA approval, it is almost universally accepted that medical marijuana has many benefits over Marinol and that by prohibiting the possession and use of natural cannabis and its cannabinoids, patients are unnecessarily restricted to use a synthetic substitute that lacks much of the therapeutic efficacy of natural cannabis. Sativex, is considered an improvement over Marinol. Sativex is an oral cannabis spray consisting of natural cannabinoid extracts, has greater bioavailability and is faster acting than oral synthetic THC. Of course, oral sprays have numerous problems as a dosage form and Saitvex has not been widely adopted as a replacement for medical marijuana. Why Marinol Is Not As Good As Real Marijuana Posted by Johnny Green on Mar. 5, 2012—see http://www.theweedblog.com/why-mannol-is-not-as-good-as-real-marijuana/ accessed Sep. 18, 2016. Incorporated by reference in its entirety.

Marinol lacks several of the therapeutic compounds available in natural cannabis. Chemical compounds in cannabis, known as cannabinoids, are responsible for its numerous therapeutic benefits. Scientists have identified 66 naturally occurring cannabinoids. The active ingredient in Marinol, synthetic delta-9-tetrahyrdocannabinol (THC), is an analogue of one such compound, THC. However, several other cannabinoids available in cannabis—in addition to naturally occurring terpenoids (oils) and flavonoids (phenols)—have also been clinically demonstrated to possess therapeutic utility. Many patients favor natural cannabis to Marinol because it includes these other therapeutically active cannabinoids. Why Marinol Is Not As Good As Real Marijuana Posted by Johnny Green on Mar. 5, 2012—see http://www.theweedblog.com/why-mannol-is-not-as-good-as-real-marijuana/ accessed Sep. 18, 2016.

Cannabidol (CBD) is a non-psychoactive cannabinoid that has been clinically demonstrated to have analgesic, antispasmodic, anxiolytic, antipsychotic, antinausea, and anti-rheumatoid arthritic properties. Clinical studies have shown CBD to possess anti-convulsant properties, particularly in the treatment of epilepsy. Natural extracts of CBD, when administered in combination with THC, significantly reduce pain, spasticity and other symptoms in multiple sclerosis (MS) patients unresponsive to standard treatment medications. CBD has been shown to be neuroprotective against glutamate neurotoxicity (i.e. stroke), cerebral infarction (localized cell death in the brain), and ethanol-induced neurotoxicity, with CBD being more protective against glutamate neurotoxicity than either ascorbate (vitamin C) or alpha-tocopherol (vitamin E). Clinical trials have also shown CBD to possess anti-tumoral properties, such as inhibiting the growth of glioma (brain tumor) cells in a dose dependent manner and selectively inducing apoptosis (programmed cell death) in malignant cells. Why Marinol Is Not As Good As Real Marijuana Posted by Johnny Green on Mar. 5, 2012—see http://www.theweedblog.com/why-mannol-is-not-as-good-as-real-marijuana/ accessed Sep. 18, 2016. Dosage formulations of CBD and other natural cannabinoids can also be formulated into solid dosage forms according to the present invention.

Additional cannabinoids possessing clinically demonstrated therapeutic properties include: cannabinol (anticonvulsant and anti-inflammatory activity); cannabichromine (anti-inflammatory and antidepressant activity); and cannabigerol (anti-tumoral and analgesic activity). Natural cannabis' essential oil components (terpenoids) exhibit anti-inflammatory properties and its flavonoids possess antioxidant activity. Emerging clinical evidence indicates that cannabinoids may slow disease progression in certain autoimmune and neurologic diseases, including multiple sclerosis (MS), Amyotrophic Lateral Sclerosis (Lou Gehrig's disease) and Huntington's Disease. Why Marinol Is Not As Good As Real Marijuana Posted by Johnny Green on Mar. 5, 2012—see http://www.theweedblog.com/why-mannol-is-not-as-good-as-real-marijuana/ accessed Sep. 18, 2016. Dosage formulations of these cannabinoids can be formulated into solid dosage forms according to the present invention.

As a result of Marinol's slow onset and poor bioavailablity, attempts are being made towards developing new formulations of pulmonary dronabinol, delivered with a pressurized metered dose inhaler. Medical News Today. “New synthetic delta-9-THC Inhaler offers safe, rapid delivery, Phase I study.” Apr. 17, 2005. Unlike oral synthetic THC, it's possible that pulmonary Marinol “could offer an alternative for patients when a fast onset of action is desirable.” Sativex, an oral cannabis spray consisting of natural cannabinoid extracts, has greater bioavailability and is faster acting than oral synthetic THC. Clinical trials comparing its bioavailability and time of peak onset compared to vaporized cannabis have not been performed, though anecdotal reports indicate that vaporized cannabis and its cannabinoids likely possess greater bioavailability and are faster acting than the Sativex spray. Thus there is a need for improved bioavailability, simple, inexpensive solid dosage forms of natural and synthetic cannabinoids.

U.S. Pat. No. 6,403,126 (incorporated herein by reference in its entirety) discloses methods of extracting and purifying cannabinoids from Cannabis using organic solvent.

An analog of dronabinol, nabilone, is available commercially.

US 20120231083 discloses a sustained release medicament which results in delivery of a therapeutic level of one or more cannabinoids. Oral administration of the disclosed compositions provides therapeutic dosing while maintaining safe, side effect sparing, levels of a cannabinoid. The present invention also provides methods of treating cannabinoid-sensitive disorders.

US 20060257463 discloses a method of transmucosally delivering a cannabinoid to a subject in need of such treatment comprising the steps of: administering to the subject a transmucosal preparation containing the cannabinoid wherein said transmucosal preparation is made by incorporating an effective amount of the cannabinoid via hot-melt extrusion technology, hot-melt molding, admixing or a solvent cast technique into a film matrix or a reservoir containing the cannabinoid, and attaching said transmucosal preparation to the mucosa of the subject.

Pharmaceutical compositions comprising the cannabinoid active pharmaceutical ingredient, crystalline trans-(±)-Δ9-tetrahydrocannabinol, and formulations thereof are disclosed in WO 2006133941. The invention also relates to methods for treating or preventing a condition such as pain comprising administering to a patient in need thereof an effective amount of crystalline trans-(±)-Δ9-tetrahydrocannabinol. In specific embodiments, the crystalline trans-(±)-Δ9-tetrahydrocannabinol administered according to the methods for treating or preventing a condition such as pain can have a purity of at least about 98% based on the total weight of cannabinoids.

U.S. Patent publication 20140100269 discloses oral cannabinoid formulations, including an aqueous-based oral dronabinol solution, that are stable at room or refrigerated temperatures and may possess improved in vivo absorption profiles with faster onset and lower inter-subject variability.

U.S. Pat. No. 8,632,825 discloses the use of a combination of cannabinoids, particularly tetrahydrocannabinol (THC) and cannabidiol (CBD), in the manufacture of a medicament for use in the treatment of cancer.

U.S. Pat. No. 6,630,507 discloses that cannabinoids have antioxidant properties. This property makes cannabinoids useful in the treatment and prophylaxis of wide variety of oxidation associated diseases, such as ischemic, age-related, inflammatory and autoimmune diseases. The cannabinoids are found to have particular application as neuroprotectants, for example in limiting neurological damage following ischemic insults, such as stroke and trauma, or in the treatment of neurodegenerative diseases, such as Alzheimer's disease, Parkinson's disease and HIV dementia. Nonpsychoactive cannabinoids, such as cannabidoil, are particularly advantageous to use because they avoid toxicity that is encountered with psychoactive cannabinoids at high doses useful in the method of the present invention.

U.S. Pat. No. 8,808,734 discloses stable, fast-acting liposomal and micelle formulations of cannabinoids or cannabinoid analogues.

U.S. Pat. No. 6,747,058 discloses stable composition for inhalation therapy comprising delta-9-tetrahydrocannabinol and semi-aqueous solvents.

DOSAGE AND ADMINISTRATION OF DRONABINOL FROM FDA DOCUMENT NDA 18-651/S-021; 500012 Rev September 2004:

Appetite Stimulation: Initially, 2.5 mg Dronabinol Capsules should be administered orally twice daily (b.i.d.), before lunch and supper. For patients unable to tolerate this 5 mg/day dosage, the dosage can be reduced to 2.5 mg/day, administered as a single dose in the evening or at bedtime. If clinically indicated and in the absence of significant adverse effects, the dosage may be gradually increased to a maximum of 20 mg/day, administered in divided oral doses. Caution should be exercised in escalating the dosage because of the increased frequency of dose-related adverse experiences at higher dosages.

Antiemetic: Best administered at an initial dose of 5 mg/m2, given 1 to 3 hours prior to the administration of chemotherapy, then every 2 to 4 hours after chemotherapy is given, for a total of 4 to 6 doses/day. Should the 5 mg/m2 dose prove to be ineffective, and in the absence of significant side effects, the dose may be escalated by 2.5 mg/m2 increments to a maximum of 15 mg/m2 per dose. Caution should be exercised in dose escalation, however, as the incidence of disturbing psychiatric symptoms increases significantly at maximum dose.

Despite all of the work on cannabinoids and dronabinol, there is a need in the art for simple, inexpensive, improved dosage forms that have an improved profile with faster onset profiles and lower inter-subject variability than currently available cannabinoid products.

According to the “Intra-Agency Agreement Between the Eunice Kennedy Shriver National Institute of Child Health and Human Development (NICHD) and the U.S. Food and Drug Administration (FDA) Oral Formulations Platform—Report 1” dronabinol is a class 2 or class 4 drug with low solubility and unknown permeability. Thus it may be formulated in the same manner as a class 2 drug.

Absorption and distribution: Dronabinol capsules are almost completely absorbed (90 to 95%) after single oral doses. Due to the combined effects of first pass hepatic metabolism only 10 to 20% of the administered dose reaches the systemic circulation. FDA document NDA 18-651/S-021.

Immediate Release or “Flash-Melt” Dosage Forms

Solid dosage forms that rapidly dissolve or disintegrate in the gastrointestinal tract or that can be dissolved prior to ingestion have been known in the art for many years. These dosage forms have the advantage of convenience and wide consumer acceptance. The need of having an oral dosage form that will rapidly dissolve or disintegrate in the mouth for situations where immediate dosing is necessary and water is not available has long been recognized.

Fast melt dosage forms are intended to dissolve or disintegrate in the mouth of the patient in one minute or even less. Rapid dissolving dosage forms are intended for primary dissolution or disintegration within less than 20 minutes in stomach acid or a container of water. The time in which a dosage form must dissolve in the mouth is much shorter than in the stomach with the obvious exception of dosage forms that are specifically formulated to slowly dissolve in the mouth.

There is an acute need for dosage forms that can rapidly dissolve or disintegrate for the benefits of having a therapeutic dosage available for absorption in a very short time. Flash-melt dosage forms are also advantageous for administration of drugs to patients such as the young, the elderly, the non-compliant and those with impairments that make it difficult to swallow an solid dosage form. Flash-melt dosage forms are also a convenience for situations where potable water may not be readily available or desirable such as sedatives, hypnotics, antipsychotics, motion sickness medication, stimulants and the like.

Cannabinoids are often taken at bed time or by cancer patients, who could greatly benefit from the flash-melt dosage forms according to the present invention.

The preparation of rapidly dissolving/disintegrating dosage forms including flash-melt dosage forms has been accomplished by freeze drying a solution or suspension of drug together with suitable excipients in water or other suitable solvents. However, freeze drying suffers from several disadvantages such as the fact that a solution or stable liquid suspension of the drug must be formed before it can be freeze dried. Aqueous solutions are not suited to formulating many drugs. In addition, the process itself is typically laborious and time-consuming, the resulting dosage forms are hygroscopic, soft, require special moisture- and impact-resistant packaging, and require careful handling prior to use. See U.S. Pat. No. 6,596,311.

Freeze drying processes have been used to prepare fast disintegrating dosage forms. Depending on the freeze drying process used, the product obtained may be characterized by a solid dried highly porous microstructure of the soluble supporting agent (i.e. mannitol, glycine, lactose, gelatins, etc.) in which the active drug is homogeneously dispersed. Although this technology produces a product which rapidly disintegrates in water or in the oral cavity, a drawback is represented by the poor physical integrity of its physical structure which severely limits further manufacturing operations such as forming blister packs. See U.S. Pat. No. 6,596,311.

Another significant drawback of the freeze drying technology in manufacturing such dosage forms is the high production costs because of the lengthy duration of each freeze drying cycle (normally from 24 to 48 hours). The complexity of the industrial plants is another important factor which prejudices the large scale use of this technology for the development of rapid disintegrating tablets. Moreover, the thermal shocks, as a direct consequence of each freeze drying cycle, might physically modify the physical-chemical properties of the outer membrane of microencapsulated particles. See U.S. Pat. No. 6,596,311.

WO 98/03064 discloses the incorporation of super disintegrants in dosage form formulations to enhance dissolution. It is disclosed therein that, for cost considerations, up to 90% of a group of super disintegrants including cross-linked cellulose, cross-linked carboxymethyl cellulose, cross-linked starch, croscarmellose alkali metal salt, crospovidone, alkali metal starch glycolate and the like can be replaced by a co-disintegrant such as diatomaceous silica, a synthetic hydrous alkaline earth metal calcium silicate and a porous hydrophilic zeolite.

Japanese patent 10114655 discloses a formulation intended for rapid dissolution in the stomach that can contain up to 30% by weight of a superdisintegrant, such as crospovidone or hydroxypropylcellulose, croscarmellose and up to 30% of a neutral or basic ingredient including magnesium aluminum metasilicate, calcium silicate, a phosphoric acid salt or a metal hydroxide. The dosage form is intended for drugs that produce a gel at acidic pH.

U.S. Pat. No. 8,518,421 and related U.S. Pat. No. 9,358,207 disclose a flash-melt pharmaceutical dosage form comprising a medicament, a superdisintegrant, a dispersing agent and a binder wherein the medicament is aripiprazole, entecavir, cefprozil, pravastatin, captopril, gatifloxacin, desquinolone, omapatrilat or irbesartan and wherein greater than 80% of said dispersing agent by weight is comprised of calcium silicate.

U.S. Pat. No. 6,596,311 discloses a formulation for preparing a fast disintegrating tablet comprising a drug in multiparticulate form, one or more water insoluble inorganic excipients, one or more disintegrants, and optionally one or more substantially water soluble excipients, the amounts of said ingredients being such as to provide a disintegration time for the tablet in the order of 75 seconds or less, typically 30 seconds or less. The water insoluble inorganic excipient are generally selected from dibasic calcium phosphate, calcium phosphate tribasic, calcium sulfate, and dicalcium sulfate.

A review of ion exchange technology is given in “Kirk-Othmer Cyclopedia of Chemical Technology” (3rd ed., Vol. 13, p. 678-705, by R. M. Wheaton and L. J. Lefevre, published by Wiley and Sons). Ion exchange is the reversible interchange of ions between a solid (ion exchange material) and a liquid in which there is no permanent change in the structure of the solid. Ion exchange is used in water treatment and also provides a method of separation in many non-water processes. It has special utility in chemical synthesis, medical research, food processing, mining, agriculture and a variety of other areas. The utility of ion exchange rests with the ability to use and reuse the ion exchange material. Ion exchange occurs in a variety of substances and it has been used on an industrial basis since circa 1910 with the introduction of water softening using natural and later, synthetic zeolites. Sulfonated coal, developed for industrial water treatment, was the first ion exchange material that was stable at low pH. The introduction of synthetic organic ion exchange resins in 1935 resulted from the synthesis of phenolic condensation products containing either sulfonic or amine groups which could be used for the reversible exchange of cations or anions. See also “DOWEX Ion Exchange Resins: Fundamentals of Ion Exchange” June 2000, brochure of Dow Liquid Separations. Form No. 177-177-01837-600QRP incorporated herein by reference.

U.S. Patent Publication 20130274262 discloses the use of polacrillin potassium as a disintegrant in stable pharmaceutical compositions comprising linezolid crystalline as well as hydroxypropylmethyl cellulose as a binder, but does not disclose flash-melt dosage forms according to the present invention.

U.S. Patent publication 20130177520 discloses the use of ion-exchange resins in order to taste mask dosage forms of bitter tasting anti-retroviral.

International patent publication WO 2009123626 discloses orodisperable formulations of phosphodiesterase-5 (PDE-5) inhibitors including polacrillin potassium, but does not disclose the formulation methods or compositions of the present cannabis-derived dosage formulations.

U.S. Pat. No. 7867515 discloses to non-effervescent, orally disintegrating solid pharmaceutical dosage forms comprising progestin and polacrillin potassium, but does not disclose the formulation methods or compositions of the present cannabis-derived dosage formulations.

U.S. Pat. No. 5,501,861 discloses a fast dissolving tablet composition containing water-soluble carbohydrate made by compression-molded method. U.S. Pat. No. 5,464,632 discloses rapidly disintegratable multiparticulate tablet, which disintegrates in the mouth in less than sixty seconds, wherein the active agent is in the form of coated microcrystals or coated or uncoated micro granules.

U.S. Pat. No. 5,762,961 discloses method of making rapidly soluble tablets comprising active ingredient, diluent, binder, and volatile salt. U.S. Pat. No. 5,919,485 discloses solid oral formulation comprising olanzapine, wherein the formulation is coated with a polymer selected from hydroxypropyl methyl cellulose, hydroxyethyl cellulose, methyl hydroxyethyl cellulose, sodium carboxymethyl cellulose, hydroxypropyl cellulose, polyvinylpyrrolidone, dimethylaminoethyl methacrylate, methyl acrylate acid ester copolymer, ethyl acrylate-methyl methacrylate copolymer, methyl cellulose, and ethyl cellulose.

U.S. Pat. No. 6,149,938 discloses a process for rapidly disintegrating tablets, containing polyalcohol, an active ingredient, edible acid, optionally, other solid components selected from the group consisting of lubricants, sweetening agents, and flavors. U.S. Pat. No. 6,316,029 discloses an oral solid dose rapidly disintegrating nanoparticulate formulation.

U.S. Pat. No. 6,350,469 discloses rapidly disintegrating tablet using methylcellulose having viscosity of >1000 centipoise and an edible calcium salt.

US 2002/0071864 discloses a tablet for oral administration, which disintegrates in the oral cavity within 60 seconds, comprising a therapeutically effective amount of an active ingredient, (ii) spray-dried mannitol (iii) crospovidone, and (iv) one or more pharmaceutically acceptable excipients, the tablet containing no microcrystalline cellulose. WO 02/47607 discloses a process for the preparation of a fast dissolving solid dosage form, comprising active agent, a cementing agent and other excipients.

WO 03/026610 discloses fast dissolving dosage form using an effervescent mixture.

WO 03/086343 discloses mouth dissolving solid dosage forms by dry granulation technique comprising pharmaceutical agent with a moisture absorber and a disintegrating agent.

WO 03/086361 discloses rapidly dispersing oral composition comprising Cannabinoid in an amount of 1 mg to 25 mg, with excipients selected from the group consisting of binder, dispersing agent, filler, glidants or lubricant and sweetener.

WO 03/103629 discloses orally administered tablet that disintegrates quickly in the oral cavity in less than 60 seconds, comprising: drug, spray-dried mannitol, microcrystalline cellulose, sodium croscarmellose and lubricant.

WO 04/091585 discloses orally disintegratable tablet comprising drug and silicified microcrystalline cellulose.

Development of dosage form depends on chemical nature of the drug and polymers, the matrix structure, swelling, diffusion, erosion, the release mechanism and the in vivo environment.

The instant invention solves these problems and provides for cannabinoid flash-melt dosage forms in a technically and economically efficient and surprising manner.

### DETAILED DESCRIPTION OF THE INVENTION

In general, the most desirable oral dosage form is a tablet, and it would be advantageous if a cannabinoid containing tablet could be made available which does not suffer from the problems of expense and the need for smoking or “edible” dosage forms. There is a need for new cheap and stable dosage formulations, especially tablets, comprising an effective dose of cannabinoids or derivatives thereof.

Another aspect the invention provides a pharmaceutical or nutraceutical composition in the form of a flash-melt tablet for oral administration comprising cannabinoid wherein said tablet is preferably formed from a pharmaceutically or even nutraceutically acceptable powder.

By “nutraceutical” is meant a composition that provides medical or health benefits, including the prevention and treatment of disease. Dietary supplements and natural health products are examples of nutraceuticals. In many places natural cannabinoids are considered nutraceuticals. Within the context of this invention it is understood that the term “drug” is used generically to include prescription and non-prescription pharmaceutical products as well as nutraceuticals including dietary supplements, natural health products, medicinal foods, drinks, candy bars with active ingredients and all other similar delivery methods whether approved or unapproved.

Viewed from another aspect the invention provides a pharmaceutical or nutraceutical tablet as hereinbefore described for use in the treatment or prophylaxis of all of the disorders that medical marijuana and drabinol is used for at the present time.

As used herein, the term “drug” includes not only pharmaceuticals but also natural medicines, alternative medicines, and dietary supplements and generally refers to all forms of cannabinoids.

Rapidly disintegrating dosage forms are also made based on special grades of sugars such as mannitol, sorbitol and the like in combination with superdisintegrants. The latter are excipients that are characterized by a special wicking capacity to channel water into the interior of the dosage form, or by rapid swelling in water, both of which act to hasten disintegration. It is also known to enhance dissolution of dosage forms by the inclusion of effervescent combinations, typically sodium bicarbonate and a weak acid, such as citric acid. As noted above, effervescent formulations require special moisture resistant packaging as even very small levels of moisture may be sufficient to initiate the effervescent reaction. Techniques, such as fluidized bed granulation, are recognized as being useful in the preparation of such formulations. Too often, however, such technologies require a specific, very costly plant including special handling equipment, controlled-humidity environments and the like. In spite of such measures, dosage forms produced by such techniques typically require moisture resistant packaging, the need to include in the packaging packets or capsules of moisture absorbing agents and the like.

BCS Class II drugs present immense challenges for oral delivery and so the flash-melt formulations of cannabinoids of the present invention represent a significant improvement over existing formulations and delivery methods of cannabinoids and involves a novel formulation method for formulating cannabinoids into tablets.

The present invention provides a deceptively simple formulation solution to the problem of formulating flash-melt versions of cannabinoids involving a few simple ingredients combined in an extremely inventive and unique way. The present invention provides tablets of cannabinoid formulations using a novel combination of cannabinoid extract resins in combination with an ion exchange resin.

Cannabinoid Extract Resin

The cannabinoid extracts of the present invention can be extracted and formulated to provide a number of sustained release combinations useful in the present invention. Of particular interest are 100 percent THC tablets, 100% CBD tablets, 10:1 THC/CBD, 1:10 THC/CBD, and 50:50 THC/CBD, although other variations of the tablets may be desirable in specific situations.

In one embodiment, additional excipients are used to diminish the residual taste of the active agents. In a further embodiment, the additional excipients do not detract from the compressibility or texture of the formulations or the dissolution properties thereof.

In one embodiment, the formulation may include flavoring agents. Flavoring agents may be any such agents known in the art, such as conventional sweetening or masking agents, such as sugars, artificial sweeteners, non-sugar natural sweeteners, citric flavors, mints or menthols and specific bitter-taste masking agents, such as citric, malic or tartaric acids. Sugars may include, for example, sucrose, mannitol, sorbitol, and xylitol. Artificial sweeteners may include, for example, aspartame, and acesulfame. Non-sugar sweeteners may include, for example, estevia.

In another embodiment, the formulation may include a combination of mannitol, sucralose, powdered flavors and starch, such as a pregelatinized starch. The combination of excipients improves the compressibility of this embodiment of the formulation. Additionally, the combination of excipients increases dispersion speeds and enhances the taste-masking effect of polacrilin potassium. In a further embodiment, mannitol crystals may be included in the formulation, thereby achieving an additional improvement in the formulation's texture and compressibility.

Optionally, other excipients affecting texture, disintegration or dispersion of the formulation may be included. Such excipients include microcrystalline cellulose,

The disintegrating agent used in accordance with the present invention is selected from crosscarmellose sodium, crospovidone, sodium starch glycolate, sodium carboxymethyl cellulose, hydroxypropyl cellulose, alginic acid, alginates, polacrilin potassium, and the like or combination thereof.

The diluents used according to the present invention include but are not limited to calcium phosphate-dibasic, cellulose-microcrystalline, cellulose powdered, calcium silicate, maltodextrin, polyols such as mannitol, sorbitol, xylitol, maltitol, starlac, silicified microcrystalline cellulose, sucrose, lactose, starch and combination thereof.

Suitable lubricants according to the present invention include but are not limited to sodium lauryl sulfate, talc, magnesium stearate, sodium stearyl fumarate, stearic acid, glyceryl behenate, hydrogenated vegetable oil, zinc stearate and suitable glidants include colloidal silicon dioxide and talc.

Suitable sweeteners according to the present invention include but are not limited to sugars such as sucrose, lactose and glucose; saccharin and salts thereof; mannitol, acesulfame potassium, glycerrhizinate salt and aspartame or mixture thereof.

Suitable flavoring agents include but are not limited to strawberry guarana, peppermint, cherry, mint, caramel, raspberry, lemon, orange, tuttifruity, banana, bubble gum, preferably strawberry guarana, juicy orange, peppermint flavor or combination thereof.

### EXAMPLES

### Example 1

Ingredients useful for 25 mg cannabinoid tablet components include 25 mg of cannabinoid resin, 250 mg of Polacrilin potassium, a large excess of mannitol and flavoring.

### Example 2

Alternative ingredients useful for 15 mg cannabinoid tablet components include 15 mg of cannabinoid resin, 4.5 mg of Polacrilin potassium, 7.5 mg of crospovidone, 1.5 mg microcrystalline cellulose, 250 mg mannitol, 1 mg colloidal silicon dioxide, 1 mg talc, 15 mg sodium stearyl fumarate; 5 mg sugar and flavoring.

### Example 3

Formulation Methods. The formulation according to the present example may be prepared as follows:

i) Cannabinoid resin, polacrilin potassium, crospovidone, microcrystalline cellulose, mannitol, colloidal silicon dioxide, talc, and flavoring are mixed in a blender,

ii) the blend obtained in step (ii) is lubricated with sodium stearyl fumarate; and

iii) the lubricated blend of step (iii) is compressed to obtain tablets.

As will be immediately obvious, some of the steps may be carried out simultaneously or in a different order, such variations are included in the present invention.

All publications mentioned above are hereby specifically incorporated herein by reference in full for the teachings for which they are cited. The examples and claims of the present invention are not limiting. Having read the present disclosure, those skilled in the art will readily recognize that numerous modifications, substitutions and variations can be made to the description without substantially deviating from the invention described herein. Such modifications, substitutions and variations constitute part of the invention described herein.

1. Rapidly disintegrating dosage form of cannabinoid with no binding agent comprising of about 1-10% cannabinoid, about 1-90% of disintegrant, about 70- 85% of filler, and about 0.4-7% of lubricant.

2. The dosage form as claimed in claim 1, further comprises sweeteners and flavoring agents.

3. The dosage form as claimed in claim 1, wherein the disintegrating agent used is selected from crosscarmellose sodium, crospovidone, sodium starch glycolate, sodium carboxymethyl cellulose, hydroxypropyl cellulose, alginic acid, alginates, polacrilin potassium or combination thereof.

4. The dosage form as claimed in claim 1 wherein the diluent is selected from calcium phosphate-dibasic, cellulose-microcrystalline, cellulose powdered, calcium silicate, maltodextrin, polyols such as mannitol, sorbitol, xylitol, maltitol, sucrose, starlac, silicified microcrystalline cellulose, lactose, starch or combination thereof.

5. The dosage form as claimed in claim 1 wherein the lubricant is selected from sodium lauryl sulfate, talc, magnesium stearate, sodium stearyl fumarate, stearic acid, glyceryl behenate, hydrogenated vegetable oil, zinc stearate and suitable glidants include colloidal silicon dioxide, talc or combination thereof.

6. The dosage form as claimed in claim 2, wherein the sweetener is selected from sugars such as sucrose, lactose and glucose; saccharin and its salts; mannitol, acesulfame potassium, glycerrhizinate salt, aspartame or combination thereof.

7. The dosage form as claimed in claim 2, wherein the flavoring agent is selected from include strawberry guarana, peppermint, cherry, mint, caramel, raspberry, lemon, orange, tuttifruity, banana, bubble gum, preferably strawberry guarana, juicy orange, peppermint flavor or combination thereof.

8. A process for the preparation of a rapidly disintegrating dosage form of cannabinoid with no binding agent comprising of about 1-10% cannabinoid, about 1-90% of disintegrant, about 70-85% of filler, and about 0.4-7% of lubricant, comprises the steps of : i) sifting cannabinoid, disintegrant, diluents, sweetener, flavoring agent, glidant, ii) mixing the sifted ingredients of step (i) in a blender, iii) lubricating the blend obtained in step (ii) and iv) compressing the lubricated blend of step (iii) to obtain orally disintegrating tablets.
